(19) United States
(12) Patent Application Publication
(10) Pub. No.: US 2022/0280730 A1
(43) Pub. Date: Sep. 8, 2022

(54) Therapeutic cooling system

(71) Applicant: PALMERA MEDICAL, INC., Mountain View, CA (US)

(72) Inventors: Jennifer Gong, San Jose, CA (US); Aaron Lee Berez, Portola Valley, CA (US); Peter Kim Nelson, Forest Hills, NY (US); Stephanie Chen, Burnaby (CA); Brent Seybold, Santa Clara, CA (US); Gregory Welsh, Los Gatos, CA (US); Thuong Dao, San Jose, CA (US); Crystal Sein Lwin, Hayward, CA (US)

(21) Appl. No.: 17/300,943

(22) Filed: Dec. 15, 2021

Related U.S. Application Data
(60) Provisional application No. 63/125,968, filed on Dec. 15, 2020.

(51) Int. Cl. A61M 5/44; A61M 25/00; A61F 7/12

(52) U.S. Cl. CPC A61M 5/44; A61M 25/0032; A61F 7/12; A61F 2007/0056

(57) ABSTRACT

A catheter system for infusing a chilled fluid into a subject can include an input lumen, a return lumen, and a transition lumen that provides sufficient flow resistance to urge a first portion of fluid flowing out of the input lumen in the forward direction to flow into the return lumen in the reverse direction toward a cooling device, while a second portion of the fluid flowing out of the input lumen in the forward direction flows out of the distal end of the catheter and into the subject's blood vessel.

### FIELD

The present disclosure relates to systems and methods for delivering fluids to an organ or vasculature of a subject.

### BACKGROUND

Therapeutic hypothermia has been shown to protect the brain from ischemia, stroke, and other acute neurological insults at the laboratory level. It has been shown to improve neurological outcome in certain clinical settings including brain injury due to cardiac arrest and neonatal hypoxia-ischemia.

However, applications of therapeutic hypothermia for stroke presents unique challenges. Unlike patients cooled following cardiac arrest, most stroke patients are awake. As a result, measures are needed to prevent shivering and discomfort experienced by deliberate cooling. Furthermore, research indicates that rapid cooling, as close to the ischemic event as possible, provides greater protection. Since cooling blankets and other external whole-body cooling techniques typically take 1-2 hours to reach mild hypothermia (33° C.), localized hypothermia may be an effective alternative.

One method to cool locally is to infuse cold saline or other suitable infusion fluid through an intravascular catheter introduced and positioned using standard interventional procedures. A flow rate of 17 ml/min is used to align with the infusion guideline of 1 liter per hour. However, the chilled fluid is warmed as it travels from the proximal hub to the distal tip due to heat exchange at body temperature (37° C.). Faster flow rates can be used to reduce this dwell time; however, care must be taken to prevent hemodilution.

Whole body cooling is often used to achieve hypothermia. However, this produces adverse side effects affecting almost all organ systems, leading potentially to cardiovascular dysfunction, immunosuppression, coagulation impairment, electrolyte imbalances, and acid/base disorders. Additionally, whole body cooling requires more time and thermal energy to reach a target temperature at a target site than would more localized body cooling.

Skin surface cooling methods such as cold rubbing, ice pads, cooling helmets, and cooling coils have been used to reduce temperature locally, but it may require at least 2 hours to reach target temperatures beneath the surface of the skin, with no necessary temperature reduction at an ischemic tissue, e.g., deep in the brain.

There is a need to develop a localized body cooling method to result in fast and selective hypothermia at an ischemic tissue, e.g., affected by vascular occlusion, with reduced effect to core body temperature and to avoid systemic side effects of generalized hypothermia.

The present disclosure is directed to overcoming these and other deficiencies in the art.

### SUMMARY OF THE DISCLOSURE

In one aspect, the present disclosure provides a catheter system for infusing a chilled fluid into a subject. In certain embodiments, the catheter system includes an outer elongate member, an inner elongate member, and a return lumen.

In certain embodiments, the outer elongate member is sized for insertion into a body cavity of a subject and has a transition section extending between, and in fluid communication with, a proximal section and a distal section of the outer elongate member.

In certain embodiments, the inner elongate member extends within the proximal section of the outer elongate member and has an input lumen permitting fluid to flow in a forward direction from a proximal end of the inner elongate member to a distal end of the outer elongate member and into the body cavity.

In certain embodiments, the return lumen extends within the proximal section of the outer elongate member and outside the input lumen and permits fluid to flow in a reverse direction from the transition section to a proximal end of the outer elongate member.

In certain embodiments, a lumen of the transition section provides sufficient flow resistance to urge a first portion of fluid flowing out of the input lumen in the forward direction to flow into the return lumen in the reverse direction while a second portion of the fluid flowing out of the input lumen in the forward direction flows out of the distal end of the outer elongate member.

In another aspect, the present disclosure provides a cooling system for delivering a chilled fluid to an organ or tissue of a subject. In certain embodiments, the cooling system includes a catheter system as described herein and a chiller that cools fluid infused into the input lumen.

In another aspect, the present disclosure provides a method of delivering chilled fluids to an organ or tissue. In certain embodiments, this method includes the steps of (i) providing a catheter system as described herein; (ii) inserting the distal end of the outer elongate member into the body cavity; and (iii) delivering a chilled fluid to an organ or tissue of the subject through the input lumen.

The catheter systems, cooling systems, and related methods of the present disclosure are designed to cool the temperature of blood flow over the target region or target area to below 35° C. In certain embodiments, it is preferred that this cooling of the blood flow is achieved with less than about 1.5 liters of fluid infused to the subject.

Various aspects of the present invention are also addressed by the following Paragraphs 1-28 and in the noted combinations thereof, as follows:

Paragraph 1: A catheter system for infusing a chilled fluid into a subject, comprising: an outer elongate member sized for insertion into a body cavity of a subject and having a transition section extending between, and in fluid communication with, a proximal section and a distal section of the outer elongate member, said distal section comprising an infusion lumen; an inner elongate member extending within the proximal section of the outer elongate member and having an input lumen permitting fluid to flow in a forward direction from a proximal end of the inner elongate member to a distal end of the outer elongate member and into the body cavity; and a return lumen extending within the proximal section of the outer elongate member and outside the input lumen and permitting fluid to flow in a reverse direction from the transition section to a proximal end of the outer elongate member, wherein a lumen of the transition section provides sufficient flow resistance to urge a first portion of fluid flowing out of the input lumen in the forward direction to flow into the return lumen in the reverse direction while a second portion of the fluid flowing out of the input lumen in the forward direction flows out of the distal end of the outer elongate member.

Paragraph 2: The catheter system according to Paragraph 1, further comprising a cooling device that cools (i) fluid entering the input lumen and (ii) fluid exiting the return lumen.

Paragraph 3: The catheter system according to Paragraph 1, wherein a distal end of the inner elongate member is located in the proximal section of the outer elongate member.

Paragraph 4: The catheter system according to Paragraph 1, wherein an inner diameter of the transition section lumen is smaller than an inner diameter of the input lumen.

Paragraph 5: The catheter system according to Paragraph 1, wherein an inner diameter of the transition section lumen distally is smaller than an inner diameter of the transition section lumen proximally.

Paragraph 6: The catheter system according to Paragraph 1, wherein an inner diameter of the transition section lumen tapers such that the inner diameter of the transition section lumen becomes progressively smaller distally than proximally.

Paragraph 7: The catheter system according to Paragraph 1, wherein the body cavity is a blood vessel.

Paragraph 8: The catheter system according to Paragraph 1, wherein the inner elongate member is unsecured within the outer elongate member.

Paragraph 9: The catheter system according to Paragraph 1, wherein the return lumen is coaxial with the input lumen.

Paragraph 10: The catheter system according to Paragraph 1, wherein the return lumen is parallel to the input lumen.

Paragraph 11: The catheter system according to Paragraph 1, wherein a cross-sectional area of the return lumen is greater than a cross-sectional area of the input lumen.

Paragraph 12: The catheter system according to Paragraph 1 further comprising a resistance member disposed within the input lumen and being effective to increase resistance to fluid flow within the input lumen.

Paragraph 13: The catheter system according to Paragraph 12, wherein the resistance member is adjustable to vary resistance to fluid flow within the input lumen.

Paragraph 14: The catheter system according to Paragraph 12, wherein the resistance member has multiple outer diameters and is adjustable such that positioning any of its outer diameters within the input lumen results in differing resistance to fluid flow within the input lumen.

Paragraph 15: The catheter system according to Paragraph 12, wherein the resistance member comprises a wire that extends at least partially along the length of the input lumen.

Paragraph 16: The catheter system according to Paragraph 1, wherein the respective cross-sectional areas of the return lumen and input lumen are configured using a recirculation-to-input area ratio parameter so as to provide a desired total flow rate of the catheter system, wherein the recirculation-to-input area ratio is defined as the cross-sectional area of the return lumen compared to the cross-sectional area of the input lumen.

Paragraph 17: The catheter system according to Paragraph 16, wherein the recirculation-to-input area ratio ranges from about 0.9 to about 2.3 for catheters having an inner diameter of about 0.085 inches or less.

Paragraph 18: The catheter system according to Paragraph 17, wherein the recirculation-to-input area ratio is about 1.3.

Paragraph 19: The catheter system according to Paragraph 1 further comprising a placement mechanism to assist in positioning the inner elongate member within the outer elongate member at a desired position so as to affect a desired flow rate of the chilled fluid into the body cavity or a target region of the subject.

Paragraph 20: The catheter system according to Paragraph 19, wherein the placement mechanism comprises marker bands fitted onto each of the inner elongate member and the outer elongate member to assist in positioning the inner elongate member within the outer elongate member at the desired position.

Paragraph 21: The catheter system according to Paragraph 19, wherein the placement mechanism comprises a tubing stop fitted onto the inner elongate member to assist in positioning the inner elongate member within the outer elongate member at the desired position.

Paragraph 22: A method of infusing a chilled fluid into a subject in need thereof, said method comprising: providing a catheter system according to any of Paragraphs 1-21; inserting the distal end of the outer elongate member of the catheter system into the subject; and infusing a chilled fluid into the subject through the input lumen of the catheter system.

Paragraph 23: A catheter system for use in infusing a chilled fluid into a subject in need thereof, wherein the catheter system according to any of Paragraphs 1-21 is provided, the distal end of the outer elongate member of the catheter system is inserted into the subject; and a chilled fluid is infused into the subject through the input lumen of the catheter system.

Paragraph 24: A cooling system for delivering a chilled fluid to an organ or tissue of a subject, comprising: a catheter system according to any of Paragraphs 1-21; and a chiller that cools fluid infused into the input lumen.

Paragraph 25: The cooling system according to Paragraph 24 further comprising a saline source, a fluid delivery pump device, and an accumulator, wherein the chiller comprises a heat exchanger.

Paragraph 26: The cooling system according to Paragraph 24 further comprising an elevated fluid source, a bubble trap, a waste container, and a fluid delivery pump device, wherein the chiller is a cooling unit that includes a bladder.

Paragraph 27: A method of delivering chilled fluids to an organ or tissue of a subject in need thereof, comprising: providing a cooling system according to Paragraph 24; inserting the distal end of the outer elongate member of the cooling system into the body cavity of the subject; and delivering a chilled fluid to an organ or tissue of the subject through the input lumen of the catheter system.

Paragraph 28: A cooling system for use in delivering chilled fluids to an organ or tissue of a subject in need thereof, wherein the cooling system according to Paragraph 24 is provided, the distal end of the outer elongate member is inserted into a body cavity of the subject; and a chilled fluid is delivered to an organ or tissue of the subject through the input lumen of the catheter system.

These and other objects, features, and advantages of this invention will become apparent from the following detailed description of the various aspects of the invention taken in conjunction with the accompanying drawings.

### BRIEF DESCRIPTION OF THE DRAWINGS

For the purpose of illustrating aspects of the present invention, there are depicted in the drawings certain embodiments of the invention. However, the invention is not limited to the precise arrangements and instrumentalities of the embodiments depicted in the drawings. Further, if provided, like reference numerals contained in the drawings are meant to identify similar or identical elements.

FIG. 1A is an illustration of one embodiment of a catheter system of the present disclosure for infusing a chilled fluid into a subject.

FIG. 1B is an illustration of one embodiment of a catheter system of the present disclosure for infusing a chilled fluid into a subject, which catheter system includes marker bands (e.g., radio-opaque marker bands) for lining up the inner elongate member within the outer elongate member for the desired or optimal flow rate of chilled fluid into a subject.

FIG. 1C is an illustration of one embodiment of a catheter system of the present disclosure for infusing a chilled fluid into a subject, which catheter system includes a tubing stop fitted on the inner elongate member to place the inner elongate member within the outer elongate member at a placement for the desired or optimal flow rate of chilled fluid into a subject.

FIG. 2 is an illustration showing a side view of the proximal, tapered, and distal portions of one embodiment of a catheter system of the present disclosure.

FIG. 3 is an illustration showing a cross-section view of one embodiment of a catheter system of the present disclosure.

FIG. 4 is an illustration showing a perspective view of one embodiment of a catheter system of the present disclosure.

FIG. 5 is an illustration of one embodiment of a catheter system of the present disclosure for infusing a chilled fluid into a subject.

FIG. 6 is a schematic of one embodiment of a cooling system of the present disclosure for delivering a chilled fluid to an organ or tissue of a subject.

FIG. 7A is a schematic of one embodiment of a cooling system of the present disclosure having the catheter system of the present disclosure incorporated with a saline source, peristaltic, accumulator, and heat exchanger.

FIG. 7B is a schematic of one embodiment of a cooling system of the present disclosure having the catheter system of the present disclosure incorporated with an elevated fluid source, a cooling unit (including a bladder), a bubble trap, a waste container, and a peristaltic pump.

FIGS. 8A-8C are illustrations showing various embodiments of a catheter system of the present disclosure having different distal tip locations of the inner elongate member (inner hypotube). FIG. 8A shows an embodiment that corresponds to a “neutral” tip location. FIG. 8B shows an embodiment that corresponds to a “positive” tip location (e.g., 1, 2, 3, 4, or 5 cm). FIG. 8C shows an embodiment that corresponds to a “negative” tip location.

FIGS. 9A-9D are scatterplot graphs of experimental results of an embodiment of a catheter system of the present disclosure showing total volume versus tip location at various pressures. FIG. 9A: Scatterplot at 20 psi. FIG. 9B: Scatterplot at 40 psi. FIG. 9C: Scatterplot at 60 psi. FIG. 9D: Scatterplot at 80 psi.

FIG. 10 is scatterplot graph of experimental results of an embodiment of a catheter system of the present disclosure showing total volume versus pressure.

FIG. 11 is a graph illustrating the effect of recirculation ratio on total flow rate in different return lumen inner diameters at various infusion flow rates.

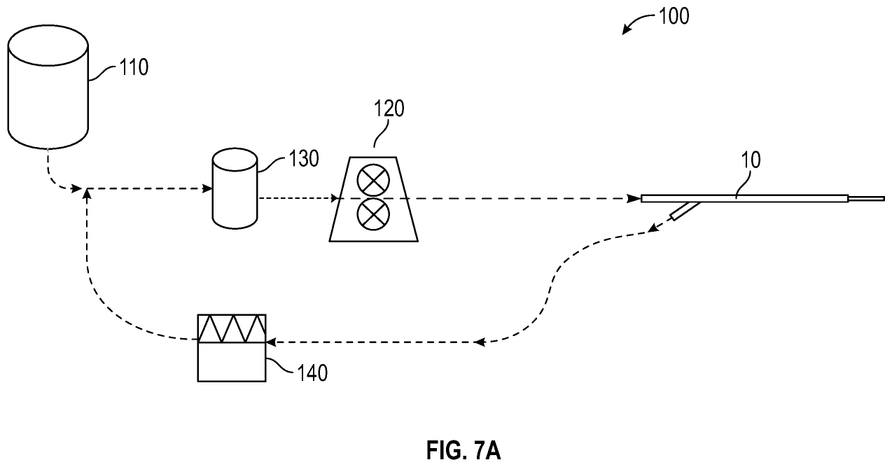

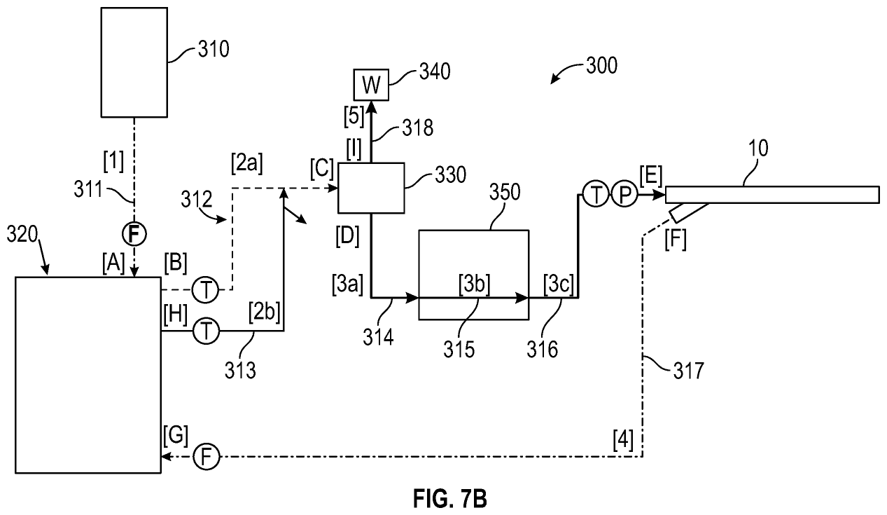

The following component list and associated numbering found in the drawings is provided to assist in the understanding of one embodiment of the present invention:

| # | Component |
|---|---|
| 10 | Catheter System |
| 20 | Outer Elongate Member |
| 21 | Transition Section |
| 21a | Inner Diameter of the Transition Section Lumen |
| 21b | Inner Diameter of the Transition Section Lumen |
| 22 | Lumen of the Transition Section |
| 23 | Proximal Section of the Outer Elongate Member |
| 23a | Proximal End of the Outer Elongate Member |
| 23b | Distal End of the Outer Elongate Member |
| 24 | Infusion Lumen: Distal Section of the Outer Elongate Member |
| 24a | Distal End of the Outer Elongate Member |
| 27 | Marker Band of the Outer Elongate Member |
| 30 | Inner Elongate Member |
| 31 | Proximal End of the Inner Elongate Member |
| 32 | Distal End of the Inner Elongate Member |
| 33 | Input lumen |
| 34 | Cross-Sectional Area of the Input lumen |
| 35 | Inner Diameter of the Input lumen |
| 37 | Marker Band of the Inner Elongate Member |
| 38a | Hemostasis Valve (e.g., Y-hemostasis valve) |
| 38b | Valve Cap |
| 39 | Tubing Stop |
| 40 | Return Lumen |
| 41 | Cross-Sectional Area of the Return Lumen |
| 42 | Resistance Member |
| 42a-42d | Outer Diameters of Resistance Member |
| 43 | Wire (e.g., guidewire) |
| 50 | Fluid |
| 51 | First Portion of Fluid |
| 52 | Second Portion of Fluid |
| 53 | Forward Direction of Fluid |
| 54 | Reverse Direction of Fluid |
| 60 | Subject |
| 61 | Body Cavity |
| 100 | Cooling System (FIG. 7A) |
| 110 | Saline Source |
| 120 | Fluid Delivery Pump Device |
| 130 | Accumulator |
| 140 | Heat Exchanger |
| 200 | Chiller |
| 300 | Cooling System (FIG. 7B) |
| 310 | Elevated Fluid Source |
| 311 | Fluid Line |
| 312 | Bladder Outlet line |
| 313 | Bladder Outlet line |
| 314 | Bubble Trap Outlet Segment |
| 315 | Pump Tubing |
| 316 | Patient Infusion Line |
| 317 | Return Line |
| 318 | Bubble Trap Vent Line |
| 320 | Cooling Unit |
| 330 | Bubble Trap |
| 340 | Waste Container |
| 350 | Fluid Delivery Pump Device |

It should be understood that the drawings are not necessarily to scale. In certain instances, details that are not necessary for an understanding of the invention or that render other details difficult to perceive may have been omitted. It should be understood, of course, that the invention is not necessarily limited to the particular embodiments illustrated herein.

### DETAILED DESCRIPTION

The present disclosure provides, inter alia, various devices, systems, and methods for infusing a chilled fluid into a subject.

As used herein, “subject” means, without limitation, a human or non-human mammal.

As used herein, “body cavity” means, without limitation, a blood vessel, bile duct, or any portion of the gastrointestinal or genitourinary tract.

As used herein, a “target portion” means, without limitation, any portion of the body of a subject to which the chilled fluid from the catheter system is to be delivered for therapeutic reasons. Examples of target portions can include, without limitation, the brain, the brain's primary motor cortex (Ml) via the middle cerebral artery (MCA), the brain's internal carotid artery (ICA), the brain's anterior cerebral artery (ACA), the liver, the kidneys, the pancreas, and/or the lungs. In certain embodiments, as used herein, the “target portion” can refer to that region of the body of a subject that is supplied by the arterial system proximal to it. For example, the MCA supplies blood to the frontal, parietal, and temporal lobes of the brain as well as the deeper structures such as the internal capsule, thalamus, and caudate nucleus. The anterior cerebral artery supplies the basal ganglia, portions of the motor cortex, and corpus callosum. The vertebral and basilar arteries may supply the cerebellum and brain stem.

As used herein, “fluid” means, without limitation, a gas or liquid, such as water or saline.

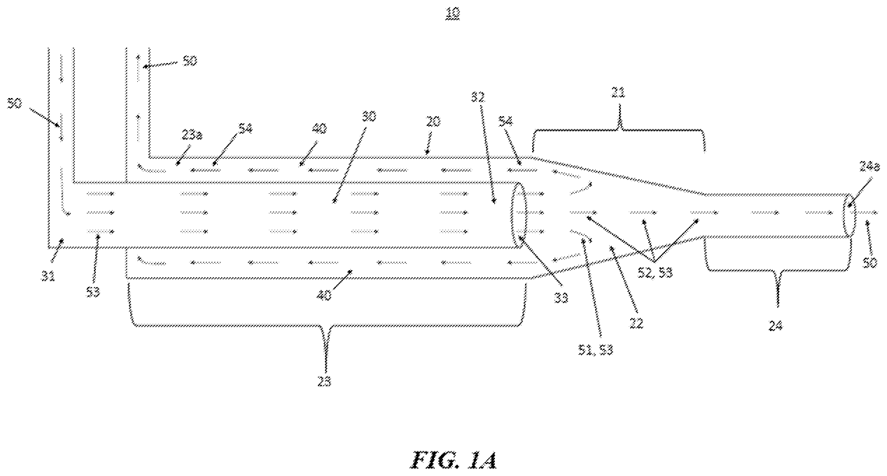

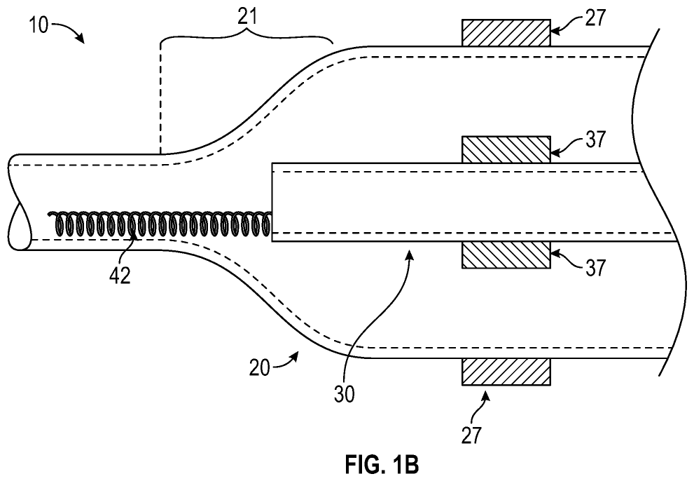

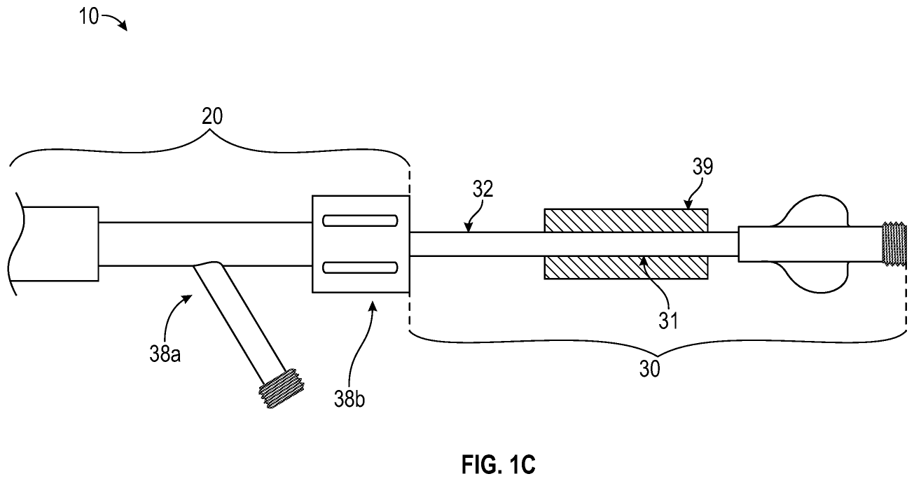

In one aspect, the present disclosure provides catheter system for infusing a chilled fluid into a subject. Turning to FIG. 1A, in certain embodiments, catheter system 10 for infusing chilled fluid 50 into a subject includes outer elongate member 20, inner elongate member 30, and return lumen 40. Outer elongate member 20 is sized for insertion into body cavity of a subject and has transition section 21 extending between, and in fluid communication with, proximal section 23 and distal section 24 (with infusion lumen) of outer elongate member 20. Inner elongate member 30 extends within proximal section 23 of outer elongate member 20 and has input lumen 33 permitting fluid 50 to flow in a forward direction 53 from proximal end 31 of inner elongate member 30 to distal end 24a of outer elongate member 20 and into body cavity (not shown). Return lumen 40 extends within proximal section 23 of outer elongate member 20 and outside input lumen 33 and permits fluid 50 to flow in a reverse direction 54 from transition section 21 to proximal end 23a of outer elongate member 20. Lumen 22 of transition section 21 provides sufficient flow resistance to urge first portion 51 of fluid flowing out of input lumen 33 in the forward direction 53 to flow into return lumen 40 in the reverse direction 54 while second portion 52 of the fluid flowing out of input lumen 33 in the forward direction flows out of distal end 24a of outer elongate member 20.

As shown in FIG. 1A, in certain embodiments of catheter system 10, distal end 32 of inner elongate member 30 is located in proximal section 23 of outer elongate member 20.

As shown in FIGS. 1B-1C, in certain embodiments of catheter system 10, outer elongate member 20 and/or inner elongate member 30 include placement mechanisms to assist in the desired placement of inner elongate member 30 within outer elongate member 20 for the desired flow rate of the cooled fluid into the body cavity or target region of the subject (e.g., the brain). The desired placement may include an optimal placement for delivering a certain rate of flow of the cooled fluid into the body cavity or target region of the subject. In certain embodiments, the optimal flow rate of the cooled fluid when it exits the catheter system is between about 16-18 mm/min of flow (e.g., when the cooled fluid exits the catheter system on its way toward a target region (e.g., the brain). The present invention also contemplates flow rates either below or above 16-18 mm/min, which may be calculated based on the teachings presented herein.

As shown in FIG. 1B, in certain embodiments, the placement mechanisms are marker bands (27, 37) that are fitted around outer elongate member 20 and inner elongate member 30. As used herein, suitable marker bands can include, without limitation, radio-opaque marker bands. In reference to the embodiment shown in FIG. 1B, to achieve the desired flow rate of fluid into the body cavity or target region of the subject, marker band 27 of outer elongate member 20 is lined up with marker band 37 of inner elongate member 30 at a predetermined stopping point within outer elongate member 20 so as to cause the cooled fluid to have the desired flow rate when exiting the catheter. In the embodiment shown in FIG. 1B, resistance member 42 is included as part of inner elongate member 30 to create a restriction to the flow to aid in accomplishing the targeted flow rate. For example, in a particular embodiment, the outer elongate member marker band 27 is placed at approximately 2 cm proximal to the transition section 21. The inner elongate member marker band 37 is placed approximately 2 cm from the distal end of the inner elongate member 32. Under fluoroscopic visioning, alignment of the two sets of marker bands will establish a recirculation-to-input area ratio parameter so as to provide the desired total flow rate of the catheter system. In certain embodiments, a suitable flow rate can include, without limitation, 16-18 mm/min of flow. The present invention also contemplates flow rates either below or above 16-18 mm/min, which may be calculated based on the teachings presented herein.

As shown in FIG. 1C, in certain embodiments of catheter system 10, the placement mechanism is a tubing stop 39 fitted on inner elongate member 30 to place inner elongate member 30 within outer elongate member 20 at a spot that achieves the desired or optimal flow rate of chilled fluid into a subject. For example, tubing stop 39 is placed on the proximal end 31 of inner elongate member 30 and prevents inner elongate member 30 from being inserted into outer elongate member 20 beyond the desired or optimal placement to achieve the desired flow rate, as well as a safety measure to prevent inner elongate member 30 from exiting outer elongate member 20. Tubing stop 39 is placed on inner elongate member 30 once outer elongate member 20 has been fully assembled, including, for example, bonding a hemostasis valve 38a (e.g., a Y-hemostasis valve) at the proximal luer. The distal end 32 of the inner elongate member 30 is aligned externally to a selected distance (e.g., approximately 2 cm) proximal to the outer elongate member's transition section, where tubing stop 39 is then bonded to inner elongate member 30 at the proximal end of the hemostasis valve cap 38b. A tooling fixture can facilitate this assembly once the initial measurements have been made. In certain embodiments, a suitable flow rate can include, without limitation, 16-18 mm/min of flow. The present invention also contemplates flow rates either below or above 16-18 mm/min, which may be calculated based on the teachings presented herein.

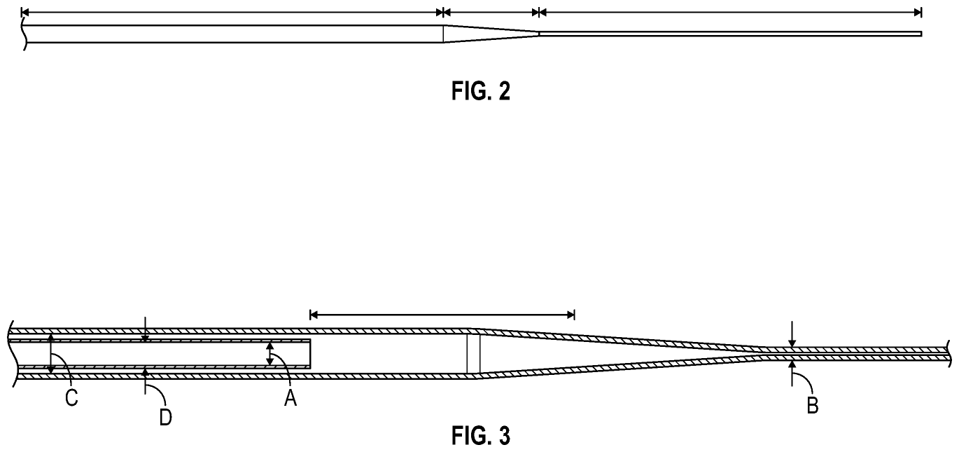

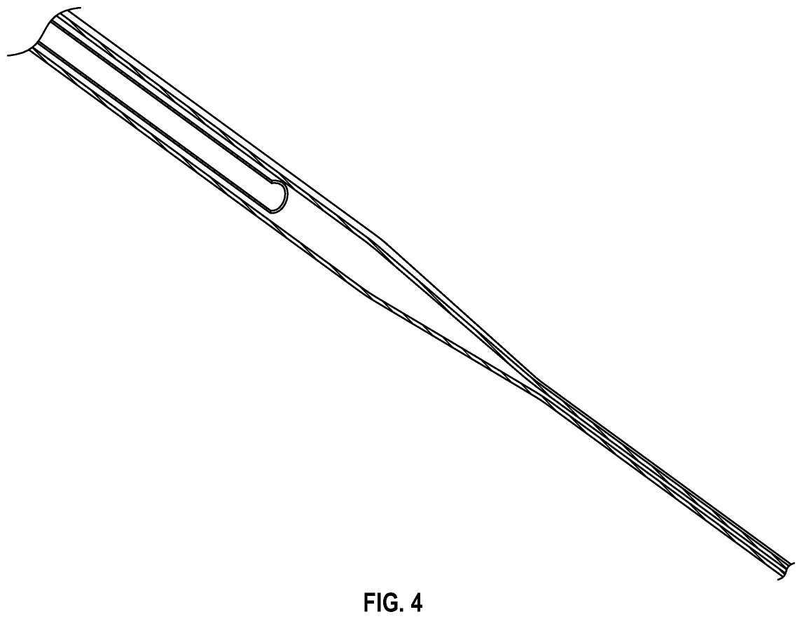

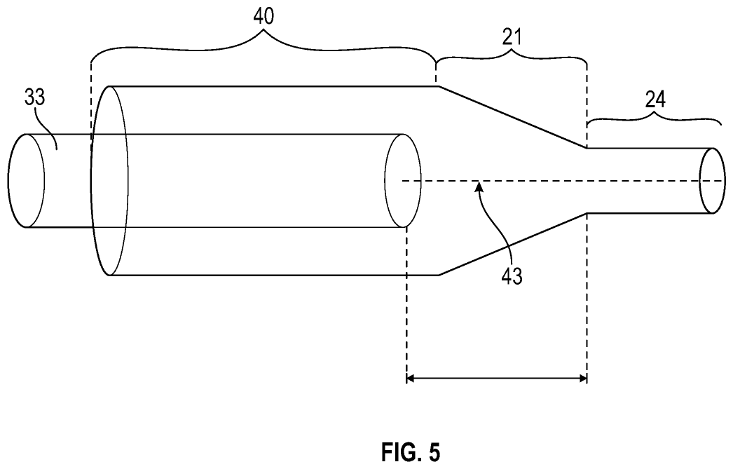

FIGS. 2-5 show various aspects of the catheter system of the present disclosure. FIG. 2 shows a side view of the proximal, tapered, and distal portions of one embodiment of a catheter system of the present disclosure. FIG. 3 shows a cross-section view of one embodiment of a catheter system of the present disclosure. FIG. 4 shows a perspective view of one embodiment of a catheter system of the present disclosure. FIG. 5 shows a catheter system of the present disclosure for infusing a chilled fluid into a subject, including the input lumen 33, return lumen 40, transition section 21, infusion lumen 24, and wire obstruction 43.

In certain embodiments of catheter system, inner diameter of transition section lumen is smaller than inner diameter of input lumen.

In certain embodiments of catheter system, inner diameter of transition section lumen distally is smaller than inner diameter of transition section lumen proximally.

In certain embodiments of catheter system, inner diameter of transition section lumen tapers such that inner diameter of transition section lumen becomes progressively smaller distally than proximally.

In certain embodiments of catheter system, inner elongate member is unsecured within outer elongate member.

In certain embodiments of catheter system, return lumen is coaxial with input lumen.

In certain embodiments of catheter system, return lumen is parallel to input lumen.

In certain embodiments of catheter system, cross-sectional area of return lumen is greater than cross-sectional area of input lumen.

In certain embodiments, catheter system further includes resistance member disposed within input lumen and being effective to increase resistance to fluid flow within input lumen.

In certain embodiments of catheter system, resistance member is adjustable to vary resistance to fluid flow within input lumen.

In certain embodiments of catheter system, resistance member has multiple outer diameters and is adjustable such that positioning any of its outer diameters within input lumen results in differing resistance to fluid flow within input lumen.

In certain embodiments of catheter system, resistance member comprises wire that extends at least partially along the length of input lumen.

In certain embodiments, the resistance member is a “flow restriction wire” or “stepped flow restriction wire.” In a particular embodiment, the resistance member can be in the form of a “guidewire” type element attached to the end of the inner lumen that could be used to regulate the flow infused out from the distal input lumen. In certain embodiments, the “stepped flow restriction wire” can have multiple steps (e.g., changes in diameter) that would allow for different infused flow rates based on which step or steps were located inside the “distal input lumen.”

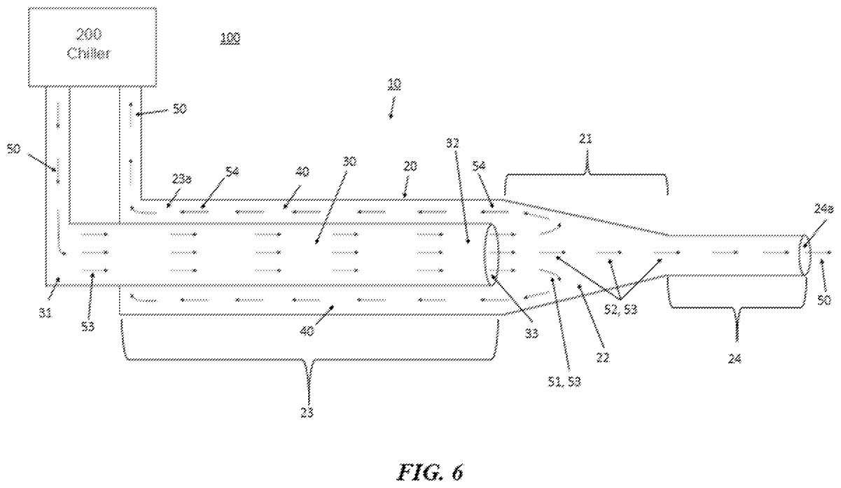

In another aspect, the present disclosure provides a cooling system for delivering a chilled fluid to an organ or tissue of a subject. Turning to FIG. 6, in certain embodiments, cooling system 100 for delivering a chilled fluid 50 to an organ or tissue of a subject (not shown), includes catheter system 10 as described herein and chiller 200 that cools fluid infused into input lumen 33.

FIG. 7A shows a cooling system 100 of the present disclosure having the catheter system 10 of the present disclosure incorporated with a saline source 110, fluid delivery pump device 120 (e.g., peristaltic device), accumulator 130, and heat exchanger 140. Suitable examples of a saline source can include, without limitation, 1 and 2 L sterile normal saline IV bags. Suitable examples of a fluid delivery pump device (e.g., a peristaltic device) can include, without limitation, Masterflex and Watson-Marlow pumps. A suitable example of an accumulator can include, without limitation, a Terumo Capiox® Bubble Trap. Suitable examples of a heat exchanger can include, without limitation, TE Technology Cold Plate Cooler and VEVOR® Recirculation Chiller Circulator Chiller.

FIG. 7B shows a cooling system 300 of the present disclosure having the catheter system 10 of the present disclosure incorporated with an elevated fluid source 310, a cooling unit 320 (including a bladder), a bubble trap 330, a waste container 340, and a fluid delivery pump device 350 (e.g., peristaltic device). Suitable examples of an elevated fluid source can include, without limitation, 1 and 2 L sterile normal saline IV bags. Suitable examples of a cooling unit (including a bladder) can include, without limitation, TE Technology Cold Plate Cooler and VEVOR® Recirculation Chiller Circulator Chiller. A suitable example of a bubble trap can include, without limitation, Terumo Capiox® Bubble Trap. Suitable examples of a waste container can include, without limitation, commonly used 2 L drainage or urinary bags. Suitable examples of a peristaltic pump can include, without limitation, Masterflex and Watson-Marlow pumps.

As shown in FIG. 7B, the tubing lines/tubing segments of cooling system 300 include fluid line 311 [1], bladder outlet lines 312 [2a] and 313 [2b], bubble trap outlet segment 314 [3a], pump tubing 315 [3b], patient infusion line 316 [3c], return line 317 [4], and bubble trap vent line 318 [5].

As shown in FIG. 7B, connection points are indicated by bracketed letters as follows: bladder fluid inlet [A], bladder fluid outlet [B], bubble trap inlet [C], bubble trap outlet [D], catheter inlet [E], catheter outlet [F], bladder return inlet [G], bladder return outlet [H], and bubble trap vent [I].

As shown in FIG. 7B, cooled saline leaves the bladder in the cooling unit at points “B” and “H”, merge together at the bubble trap, point “C”, is pumped through the peristaltic pump, point “3b”, then to the catheter at point “E”. Temperature “T” and pressure “P” are measured right at the proximal end of the catheter at the luer hub. Typically, this is 4° C. In certain embodiments, temperatures are typically of −1 to +1 degrees Celsius as the cooled fluid exits at the bladder. In certain embodiments, a thermocouple can be put into the saline stream that exits the catheter tip (e.g., the catheter is surrounded by body-heated flowing water) at about 18 cc/min, that warms the saline stream to typically measures 11° C. right at the tip. In certain embodiments, approximately 1-2 inches from the tip, the cooled saline mixed with flowing blood is typically at 30-34° C. The present disclosure contemplates the use of different measurements than described above, which can be changed with cooled saline flow rate, simulated ICA and aortic flowrate, saline temp, pressure, etc.

In another aspect, the present disclosure provides a method of delivering chilled fluids to an organ or tissue. In certain embodiments, this method includes: providing catheter system as described herein; inserting distal end of outer elongate member into body cavity; and delivering a chilled fluid 50 to an organ or tissue of subject through input lumen.

As described in more detail below, in certain applications, the catheter system, cooling system, and method of the present disclosure can be used for delivering chilled fluids to points within the vasculature, more specifically the neuro-vasculature, of a subject.

In order to cool an organ or specific site within the vascular, one must overcome the issue of the cooling medium warming as it travels to the site to be cooled. This becomes more difficult as the rate of fluid delivery (ml/min) decreases and/or the distance traveled through the vasculature increases. Increasing the flowrate would reduce the transit time to the desired site, thus allowing the fluid to arrive at a lower temperature. But as infusion rate increases so does the total volume of fluid infused (in the same amount of time), which can be undesirable.

The catheter system and cooling system of the present disclosure invention provide a means for providing the coldest possible fluid to the infusion site, by minimizing the heat transfer into the fluid being infused, without needing to increase the rate of infusion.

In certain embodiments, the catheter system of the present disclosure includes a catheter with coaxial lumens. As discussed herein, the catheter system includes an “Input” lumen which is the inner most lumen and a “Return” lumen that surrounds the input lumen. At the distal end of both lumens is a common lumen where the lumens merge and an additional lumen, the “Infusion Lumen” joins. The common lumen creates a location where the fluid from the “Input” lumen has a chance to (i) return via the return lumen or (ii) be infused into the patient via the “Infusion” lumen. One key feature of the catheter system of the present disclosure is the “Return” lumen. The Common Lumen and “Return” lumen allow the catheter system to have high flowrates of the cooled fluid, thus decreasing transit time and heat transfer, while not dictating the amount of fluid infused into the patient. The volume of fluid infused is controlled by the “Infusion” lumen. The “Return” lumen also acts as an insulator for the “Input” lumen as any heat absorbed by the fluid in the “Return” lumen is taken out of the body and not transferred to the fluid in the “Input” lumen.

Additional designs and features of the catheter system of the present disclosure are described below.

Various features of the catheter system of the present disclosure are effective to deliver the coldest infusion fluid intravascularly from the most proximal end, which may originate from the femoral artery, to the most distal end, which may terminate in the neurovasculature. In certain embodiments, thermal insulating tubing may be used to reduce warming of the infusion fluid by minimizing heat exchange from the body. A coaxial design with the infusion fluid delivered through the inner lumen may be used to further reduce warming of the infusion fluid. High infusion flow rates may be used to reduce warming of the infusion fluid by minimizing dwell time.

To deliver the coldest infusion fluid intravascularly without causing hemodilution various configurations of the catheter system of the present disclosure can be used. In some embodiments, an infusion rate of about 17 ml/min may be optimal. Incorporating a section (“pre-cooling section”) along the catheter that is proximal to the infusion site to intentionally lower the blood temperature via heat exchange may also be employed. For neurovascular applications, positioning said section distal to the bifurcation of the internal and external carotid may be optimal to reduce the induction of shivering. Positioning said section within the 30 cm most distal end of the catheter may be optimal in certain embodiments.

To use high flow rates to deliver the coldest infusion fluid intravascularly without causing hemodilution, various configures can be used, as described below.

In certain embodiments, the catheter system can be configured to recirculate a partial of the total fluid volume in the catheter. A coaxial design with the input fluid traveling through the inner lumen and the return fluid traveling through the outer lumen can be used. The outer lumen and return fluid may also serve as thermal insulation for the input fluid. The outer lumen may be straight or tapered. The inner lumen may be unsecured within the outer lumen. The distal end of the inner lumen terminating before the distal end of outer lumen may be optimal in certain embodiments. A return lumen cross-sectional area greater than input lumen cross-sectional area may be ideal in certain embodiments.

In certain embodiments, the catheter system can be configured to infuse a partial of the total fluid volume in the catheter. Decreasing the diameter of the infusion lumen may be used to reduce the fluid volume infused. Decreasing the diameter of the infusion lumen may be performed before or after the catheter is positioned within the vasculature. The infusion lumen may be straight or tapered. An infusion lumen cross-sectional area that is smaller than the input lumen cross-sectional area may be used in certain embodiments. An infusion lumen cross-sectional area that is smaller than the return lumen cross-sectional area may be used in certain embodiments.

A permanent or temporary obstruction may be placed within the catheter to reduce the infusion lumen diameter. The obstruction may be flexible, rigid, and may obstruct the entire length of the infusion lumen. In certain embodiments, the obstruction may be a wire. The obstruction may also have temperature-sensing capabilities. The obstruction may partially obstruct the length of the infusion lumen. The length of the obstruction may be used to vary the infusion flow rate.

A permanent or temporary nozzle may be placed within the catheter to reduce the infusion lumen diameter.

A permanent or temporary funnel may be placed within the catheter to reduce the infusion lumen diameter.

A hydrophilic coating on the infusion lumen may be used to reduce the infusion lumen diameter.

An inflatable balloon-like structure on the infusion lumen may be used to reduce the infusion lumen diameter.

A temperature dependent shape memory component may be used to reduce the infusion lumen diameter.

To use high flow rates to deliver the coldest infusion fluid intravascularly at the low input pressures, the catheter system of the present disclosure may be configured for applying vacuum pressure to the return lumen may increase flow rate without increasing input pressure.

To access the neurovasculature while maintaining high catheter flow rates, ideal infusion flow rates, and safe input pressures can be used. A transition length of 2-6 cm from the return lumen to the infusion lumen may be optimal in certain embodiments.

Some neuroprotective effects of hypothermia can be attributed to a reduction in oxygen demand. A decrease in brain temperature by 1° C. lowers cerebral oxygen consumption by ˜5%, thus increasing tolerance to ischemic conditions. Additionally, cooling the brain may stop or decrease some of the inflammatory and other changes initiated by the ischemia. Similarly, hypothermia can be beneficial to other ischemic tissue affected by a vascular occlusion, e.g., slowed tissue damage and improved recovery of the patient. The methods and apparatus provided herein may be used for localized body cooling to result in fast and selective hypothermia at target ischemic tissue to protect and/or improve recovery of the ischemic tissue.

Provided herein are methods comprising introducing a fluid at a location in a lumen of an artery in a subject (e.g., human, mammals), the location being downstream of an occlusion (e.g., a thrombus, or clot) in the artery, and the fluid being cooler than a blood temperature in the artery. In certain embodiments, the method further comprises: before the introducing, passing within the lumen of the artery a distal end of an elongate member from a location upstream of the occlusion to a location downstream of the occlusion in the lumen of the artery. In certain embodiments, damage to ischemic tissue downstream of the occlusion is reduced or slowed down due to the hypothermia resulting from the introducing of the fluid cooler than the blood temperature in the artery.

Provided herein are also methods comprising introducing a fluid at a location in a lumen of a vein in a subject (e.g., human, mammals), the location being upstream of an occlusion in the vein, and the fluid being cooler than the blood temperature in the vein. In certain embodiments, the method further comprises: before the introducing, passing within the lumen of the vein a distal end of an elongate member from a location downstream of the occlusion to the location upstream of the occlusion in the lumen of the vein. In certain embodiments, damage to ischemic tissue upstream of the occlusion is reduced or slowed down due to the hypothermia resulted from the introducing of the fluid cooler than the blood temperature in the vein.

As used herein, an “occlusion” is a partial or total obstruction, e.g., of a blood vessel, such as an artery or vein.

As used herein, “hypothermia” means that a tissue or organ temperature (e.g., of ischemic tissue) in a subject is at least 1° C. lower than core temperature or than blood temperature in a vein or artery of the subject. A localized hypothermia can be beneficial for protecting tissues. For example, a woman survived without brain damage after being trapped under ice for over an hour, when her core temperature reportedly dropped to about 13.7° C.

A person skilled in the art, such as a medical practitioner, would be able to achieve local hypothermia beneficial to a subject treated by adjusting or choosing the temperature of cold fluid introduced into the subject's blood vessel based on one or more of various factors, e.g., the flow rate of the cool fluid introduced; the composition of the cool fluid introduced; the size, location, and metabolic rate of any ischemic tissue that may benefit from hypothermia; the location and anatomy of the occluded vessel; the rapidity of induction of hypothermia; the patient's physical condition; and other comorbidities.

The catheter systems, cooling systems, and related methods of the present disclosure are designed to cool the temperature of blood flow over the target region or target area to below 35° C. In certain embodiments, it is preferred that this cooling of the blood flow is achieved with less than about 1.5 liters of fluid infused to the subject. In certain embodiments, a infusion fluid (e.g., saline) is cooled down to about −1° C. at the cooling unit (e.g., heat exchanger), it warms to about 4° C. at the hub of the inner elongate member (i.e., inner catheter) and exits the outer elongate member (i.e., outer catheter) into the blood flow at about 11° C. with a flow rate of between about 16 and 18 cc/min. In certain embodiments, at 2 cm distal to the distal end of the outer elongate member (outer catheter) tip, the cooled fluid (e.g., saline) has mixed with blood flow, thereby bringing the temperature of the mixture to between about 30-34° C. The present disclosure contemplates the use of insulation to insulate the cooling system between the heat exchanger and catheter hub and having less warming within the inner elongate member (inner catheter) while it is in the body of the subject to further drop the exit temperatures.

In certain embodiments of the methods disclosed herein, the fluid introduced has an inlet temperature of about 2° C. and an exit temperature of about 35° C. In embodiments, the inlet temperature of the fluid may be greater than or less than 2° C. In certain embodiments, the temperature of the fluid exiting the catheter system may range from about 5-15° C. In certain embodiments, the fluid is introduced until completion of a thrombectomy or other appropriate procedure for removal of the occlusion. In some embodiments, the fluid is introduced for an extra 30-60 minutes, 1-3 hours, 6-12 hours 12-24 hours, 1-3 days, or other period depending on patient response or other factors. In certain embodiments, the fluid is introduced as soon as possible after symptoms or signs of the occlusion occur, e.g., within about 1, 2, or 4 hours, within about 6-12 hours, within about 12-24 hours, within about 1-3 days, or within 7 days after symptoms or signs of the occlusion occur. As used herein, the temperature of the fluid introduced can either be the temperature of the fluid as it exits the catheter or the temperature of the fluid/blood mixture after the fluid exits the catheter and mixes with the subject's blood.

As used herein, “catheter” has its ordinary meaning and can include any elongate structure, such as a tubular member, configured to transmit fluid or objects through a conduit extending along at least a portion of the catheter's length. A catheter may have any of many cross sectional shapes, such as round or polygonal and may resemble a tube, ribbon, etc. As used herein, “guide wire” (or “guidewire”) has its ordinary meaning and can include any elongate structure, such as metallic and/or polymeric member, configured to extend into a body viscus or vessel to facilitate access to a location in the body by a catheter or other device. A guide wire may have any of many cross sectional shapes, such as round or polygonal and may resemble a wire, ribbon, rope, or other object. As used herein, peristaltic pump has its ordinary meaning and can include any fluid delivery device, such as diaphragm pumps, gear pumps, centrifugal pumps, configured to provide the system with adequate fluid delivery within the operating pressures.

In certain embodiments of the methods disclosed herein, the introducing the fluid is through a catheter, an elongate member passing the occlusion. In certain embodiments, the introducing the fluid is through a second elongate member (catheter), different from the first elongate member passing the occlusion (a guide wire). In certain embodiments, the catheter may comprise a plurality of lumens (see, e.g., examples shown in FIGS. 1-5) for introducing different fluids, and/or introducing other accessories as desired, e.g., guidewire, an expandable element (e.g., balloon, also referred to herein as an expandable member), stent, drilling element (e.g., by ultrasound), imaging element, retrieval element, cooling element (also referred to as a thermal member herein), thermally insulate element, sensor element, and any combinations thereof, as described in this disclosure.

The cooling catheter of the disclosure is, in some embodiments, placed intra-arterially with its distal tip in the internal carotid artery (ICA). This provides a thermally insulated conduit to the ICA. The cooling catheter may be flushed with cold flush solution during the procedure.

In certain embodiments of the methods disclosed herein, the passing includes passing through or around a portion of the occlusion. In certain embodiments, the elongate member comprises a penetrating element (e.g., an element with a blunt or sharp distal end, and/or with a drilling element at the distal end) passing through or around the portion of the occlusion. The penetrating element may be pulled out from the catheter after the passing step such that the fluid can be introduced through the elongate member. For catheters having a plurality of lumens, the penetrating element may not need to be pulled out before the introducing the fluid, as the fluid may be introduced through a different lumen.

In certain embodiments of the methods disclosed herein, the method further comprises utilizing the one or more lumens of the catheter, and/or one or more accessories for one or more tasks these lumens and/or accessories may be used for. For example, the method may further comprise one or more steps of retrieving the occlusion via the retrieval element, providing image of the location downstream of the occlusion of an artery or upstream of the occlusion of a vein, detecting one or more parameters of the location downstream of the occlusion of an artery or upstream of the occlusion of a vein by one or more sensor elements, and cooling the fluid until it is introduced to the desired location.

In certain embodiments of the methods disclosed herein, the passing includes passing around the occlusion, e.g., between the occlusion and the vessel wall of the artery or vein. In certain embodiments, the elongate member comprises a guidewire that passes between the occlusion and the vessel wall of the artery or vein. The elongate member may be a catheter comprising one or more lumens. The guidewire may be pulled out from the catheter such that the fluid can be introduced through the catheter. For catheters having a plurality of lumens, the guidewire may not need to be pulled out before the introducing the fluid, as the fluid may be introduced through a different lumen.

In certain embodiments of the methods disclosed herein, a catheter may be configured to comprise an expandable element (e.g., balloon) close to the distal end of the catheter, and the method further comprises expanding the expandable element after the distal end of the elongate member and the expandable element are positioned at a desired location. The expandable element can occlude the blood vessel for flow arrest while introducing the cooler fluid.

In certain embodiments of the methods disclosed herein, the occlusion can be removed by a retrieval element the catheter is configured with, either immediately or after having first cooled the ischemic tissue to the desired therapeutic hypothermic temperature. For example, the retrieval element may be positioned adjacent to an occluding thrombus and retrieve the thrombus. Examples of retrieval elements include, without limitation, a thrombectomy device (e.g., Solitaire® revascularization device), basket, wire, or atherectomy device.

In certain embodiments of the methods disclosed herein, the fluid is cooled extracorporeally. In certain embodiments, the fluid is cooled or maintained at a temperature lower than the blood temperature of the subject treated when a catheter introducing the fluid comprises a cooling mechanism and/or a thermal insulator.

In certain embodiments of the methods disclosed herein, the fluid comprises an intravenous solution. Examples of the intravenous solutions include, without limitation, colloid solutions, crystalloids, and blood products such as serum or plasma. Further examples of the colloid solutions include, without limitation, albumin (e.g., 5% or 25%), hetastarch (hespan), dextran. Examples of the crystalloid solutions include, without limitation, normal saline, half normal saline, lactate ringers, and dextrose 5%, D5 half-normal saline. In certain embodiments, the fluid may further comprise additional oxygen dissolved therein.

In certain embodiments of the methods disclosed herein, the fluid further comprises one or more active ingredients (AI) for therapeutic and/or diagnostic purposes. Examples of the AI may include thrombolytic agents such as tissue plasminogen activator (tPA), streptokinase, or urokinase. The AI may slow down apoptosis and/or metabolism of ischemic tissue either downstream of the occlusion in an artery or upstream of the occlusion in a vein. For example, kinase inhibitors (e.g., tyrosine kinase inhibitors, GSK-3 inhibitors, PI3-kinase gamma inhibitors), monocarboxylate transporter (MCT) inhibitors may be used. Ms may also include osmotic agents such as mannitol (e.g., 20% mannitol) to reduce intracranial pressure, and any combinations of agents or classes of agents.

In certain embodiments of the methods disclosed herein, the occlusion in the artery or vein can include a thrombus, dissection, atheromatous plaque, embolism (by air, fat, foreign body, thrombus), or any combinations thereof. The organs the occlusion in the artery or vein may affect include, without limitation, brain (e.g., thrombotic or thromboembolic stroke, certain cases of hemorrhagic stroke, traumatic brain injury, and iatrogenic injury during interventional procedures), heart, lung, limbs, liver, pancreas, spleen, and kidney.

There is a limitation in delivering cold fluid to a lesion site. Cold fluid injected in the hub of a regular catheter that is placed in warm, flowing blood is warmed by the time it reaches the tip of the catheter. Provided herein are catheters comprising a lumen for introducing the cooler fluid to a location desired as described in the methods herein.

In certain embodiments, the catheter comprises a delivery lumen through which the cooler fluid travels through until introduced to a desired location as described herein. In certain embodiments, the delivery lumen is thermally insulated (a thermally insulated lumen), which reduces the temperature change of the fluid traveling through. Thereby, the cooler fluid exiting from the distal end of the delivery lumen can cool down at least a portion of the tissue the cooler fluid contacts. In certain embodiments, the tissue is in a brain, heart, lung, limbs, or kidney, and such delivery of cooler fluid as disclosed herein may lower the temperature of the organ and induce therapeutic hypothermia to the ischemic tissue affected by the artery with the occlusion.

In certain embodiments, the catheter comprises a cooling element not only reducing the temperature gain of a cooler fluid traveling through, but also further lowering the temperature of the fluid. The cooling element may comprise a heat transfer medium circulation system, wherein a heat transfer medium is circulated to provide heat exchange through the inside wall of the catheter where the fluid travels through. The cooling element may comprise a compressed air cooling system or any other cooling system that can implement the desired heat exchange with the fluid.

For example, the cooling element can surround a substantial portion of the delivery lumen of the catheter that is close to the distal end of the catheter and is in contact with blood in the blood vessel the catheter travels through. The cooling element comprises a plurality of ports for a plurality of lumens in desired fluid communication for circulation with a chiller unit providing cooler heat transfer media, a cooler media suitable for heat transfer can be introduced into the cooling element from one or more of the ports, then travels through the corresponding lumens until exiting the cooling element from one or more of the ports. The fluid traveling through the delivery lumen is cooled or maintained cool when in heat transfer with the cooler heat transfer media circulated in the plurality of lumens of the cooling element.

In certain embodiments, the catheter further comprises an expandable element close to its distal end such that once the expandable element passes through the occlusion as described herein, it has a diameter larger than the majority section of the catheter from its proximate end. The larger diameter of the expandable element prevents the fluid introduced through the catheter from backflow. For example, the largest diameter of the expandable element may be about 2 to about 10 times larger, about 2 to about 15 times, or about 2 to about 20 times larger than the diameter of the catheter section immediately following the expandable element.

In certain embodiments, the expandable element expands after passing through the occlusion as described herein. Examples of expandable elements include, without limitation, balloons.

In certain embodiments, a distal access catheter may be placed through the delivery lumen, e.g., the distal access catheter may be essentially a large bore (e.g., 5 or 6F catheter about 0.045″ to 0.07″ ID) with its tip going more distal. If the tip of the distal access catheter can reach the occlusion (e.g., thrombus) it can potentially be used as an aspiration catheter to remove the occlusion. The distal access catheter can be flushed with the cooler fluid.

Through the delivery lumen of the distal access catheter, a microcatheter (e.g., 0.021 or 0.027″ ID) can be passed over a guide wire and the tip of the microcatheter can be advanced carefully past the occlusion. Once the distal end of the distal access or the delivery lumen is positioned in a desired location in the blood vessel, cooling of the tissues affected by the occlusion (e.g., ischemic parenchyma) can begin immediately by introducing the cooler fluid as disclosed herein, even before attempted removal of the occlusion and restoration of flow. Cooling the ischemic tissue prior to the restoration of the flow of oxygenated blood may decrease reperfusion injury. The ischemic tissue will be cooled by infusion of cooler fluid as disclosed herein, which may increase oxygen carrying capacity (e.g., by blood or artificial heme) and/or include AIs as disclosed herein to decrease the size of the infarct.

Additionally, a retrieval device as described herein may be brought adjacent to the occlusion by passing through the lumen of a microcatheter, then unsheathing or otherwise deploying the retrieval device, and retrieving the occlusion.

### EXAMPLES

The following examples are intended to illustrate particular embodiments of the present disclosure, but are by no means intended to limit the scope of the present disclosure.

### Example 1

In certain embodiments, a faster flow rate can be used to reduce the dwell time without surpassing the hemodilution limit if most of the fluid is recirculated and only a portion of the total fluid volume is infused. To achieve this, a coaxial design was explored, where the cold saline was delivered through the inner lumen and the recirculation path was on the outer lumen. For these experiments, hypotubes in various diameter combinations were used to simulate the proximal end and a needle valve was used on the distal end to simulate the infusion lumen and control the infusion flow rate. A return lumen cross-sectional area greater than the input lumen cross-sectional area yielded more recirculated fluid. Additional experiments exploring the effects of fluid input pressure, including vacuum pressure, were also performed.

For all experiments, the distal end of the inner lumen was not fixed radially.

Experimentation to determine if the longitudinal position of the inner lumen to the outer lumen affected recirculation flow rate was performed, and results indicated that the distal end of the inner lumen should terminate at least 2 cm proximal to the infusion lumen for optimum recirculation.

After determining the input-to-return ratio, various lumen diameters were tested to determine the infusion lumen diameter that yielded the target flow rate. It was observed that an infusion lumen diameter smaller than the input lumen diameter was ideal to balance recirculation and infusion, with an infusion lumen diameter of 0.009″ to 0.010″ that achieved the target flow rate. However, these diameters are too small to be compatible with commonly used neurovascular guidewires.

The infusion lumen diameter was increased to be compatible with neurovascular guidewires, and experimentation on methods to restrict the infusion diameter after the catheter is positioned at the target location were explored. One method to reduce the infusion diameter, thus reducing infusion flow rate, is using a wire to obstruct a portion of the infusion lumen. Various wire diameters were tested to trend the relationship between the wire diameters and infusion flow rate.

### Example 2

Inner Hypotube Tip Location Experiment

Purpose: To determine various flow characteristics of different hypotubes with regards to the distal tip locations.

Test Setup: A Bronkhorst NV-013-HR valve was used to regulate the flow out of the distal tip.

Test Methodology: The distance between the distal tip of the outer and inner hypotube was varied. Testing was performed at 20, 40, 60, and 80 psi with distal flow regulated to 18 mL/min±2 mL/min. No vacuum was applied.

Dimensions: Outer hypotube: Inner Diameter (ID): 0.071 inches. Inner hypotube: Outer Diameter (OD): 0.050 inches; Inner Diameter (ID): 0.038 inches. The design of the hypotube used in this experiment is shown in FIG. 6. As used in this example, tip location is measured as follows: (i) a measurement of 0.0 cm means that the tips of the outer hypotube and inner hypotube are aligned; (ii) a negative measurement (e.g., −1, −2, −3, −4, or −5 cm) describes the distance that the tip of the inner hypotube is retracted from the tip of the outer hypotube; and (iii) a positive measurement (e.g., 1, 2, 3, 4, or 5 cm) describes the distance that the tip of the inner hypotube protrudes from the tip of the outer hypotube.

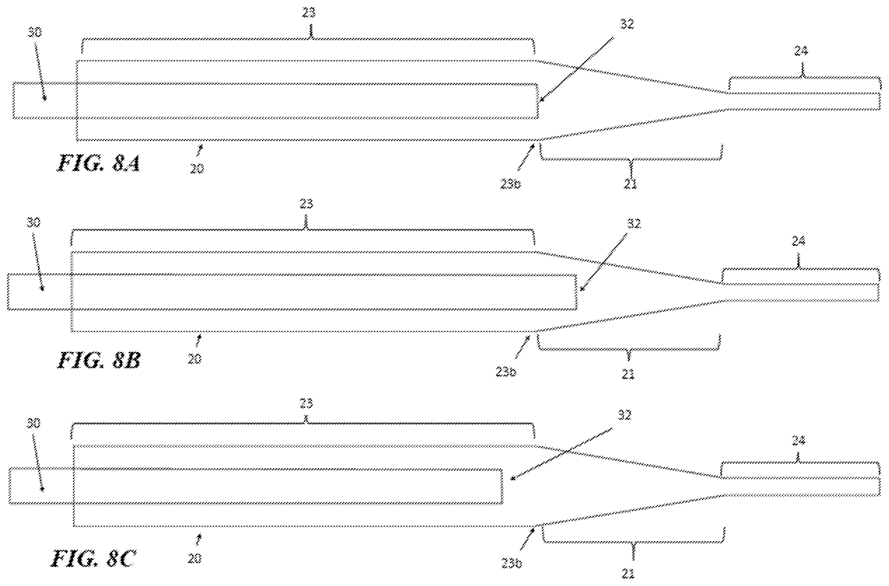

FIGS. 8A-8C illustrate embodiments of a catheter system of the present disclosure having different distal tip locations of the inner elongate member (inner hypotube). FIG. 8A shows an embodiment that corresponds to a “neutral” tip location (e.g., 0.0 cm), where the distal end (32) of the inner elongate member (30) is aligned with the distal end (23b) of the proximal section (23) of the outer elongate member (20). FIG. 8B shows an embodiment that corresponds to a “positive” tip location (e.g., 1, 2, 3, 4, or 5 cm), where the distal end (32) of the inner elongate member (30) protrudes beyond the distal end (23b) of the proximal section (23) of the outer elongate member (20). FIG. 8C shows an embodiment that corresponds to a “negative” tip location (e.g., −1, −2, −3, −4, or −5 cm), where the distal end (32) of the inner elongate member (30) is retracted into the distal end (23b) of the proximal section (23) of the outer elongate member (20).

Results: Return flow and total flow at the various pressures, tip locations, and tip flow were measured, as shown in Table 1 below.

TABLE 1

| Pres- | Tip | Tip | Return | Total |
|---|---|---|---|---|
| sure | Location | Flow | Flow | Flow |
| (Psi) | (cm) | (mL/min) | (mL/min) | (mL/min) |
| 20 | −5 | 18 | 47 | 65 |
| 20 | −4 | 18 | 47 | 65 |
| 20 | −3 | 17.5 | 48 | 65.5 |
| 20 | −2 | 17 | 51 | 68 |
| 20 | −1 | 18 | 48 | 66 |
| 20 | 0 | 18 | 46 | 64 |
| 20 | 1 | 17 | 45 | 62 |
| 40 | −5 | 18.5 | 84 | 102.5 |
| 40 | −4 | 18.5 | 84 | 102.5 |
| 40 | −3 | 17.5 | 85 | 102.5 |
| 40 | −2 | 17 | 87 | 104 |
| 40 | −1 | 18.5 | 84 | 102.5 |
| 40 | 0 | 17.5 | 84 | 101.5 |
| 40 | 1 | 18.5 | 81 | 99.5 |
| 60 | −5 | 19 | 106 | 125 |
| 60 | −4 | 19 | 106 | 125 |
| 60 | −3 | 19 | 107 | 126 |
| 60 | −2 | 19 | 110 | 129 |
| 60 | −1 | 19 | 107 | 126 |
| 60 | 0 | 17.5 | 106 | 123.5 |
| 60 | 1 | 18 | 104 | 122 |
| 80 | −5 | 18.5 | 129 | 147.5 |
| 80 | −4 | 19 | 129 | 148 |
| 80 | −3 | 18 | 129 | 147 |
| 80 | −2 | 18.5 | 135 | 153.5 |
| 80 | −1 | 18 | 129 | 147 |
| 80 | 0 | 18 | 125 | 143 |
| 80 | 1 | 18 | 125 | 143 |

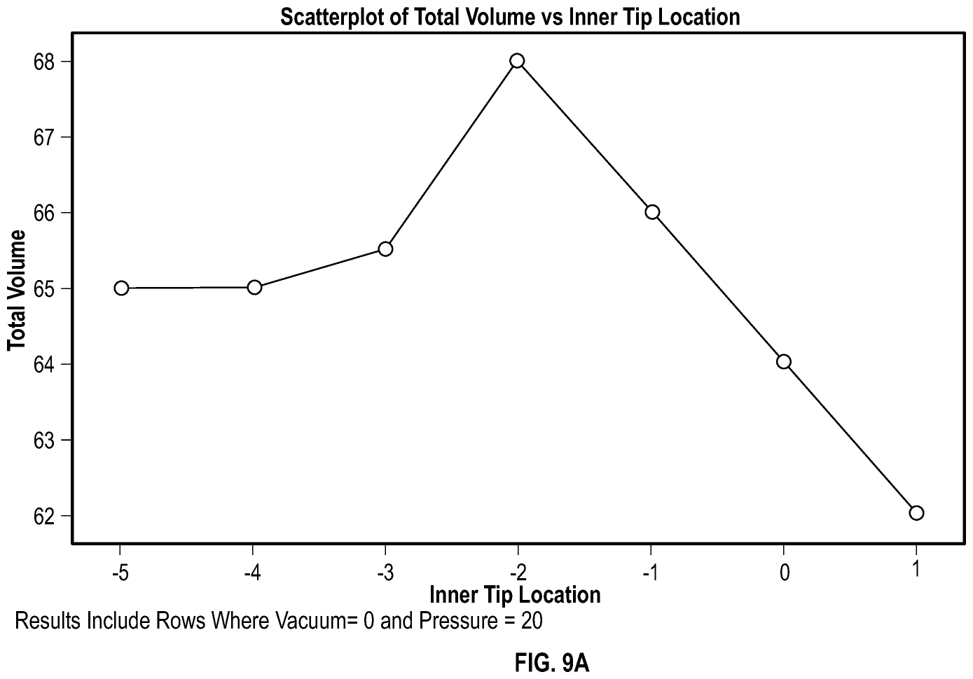

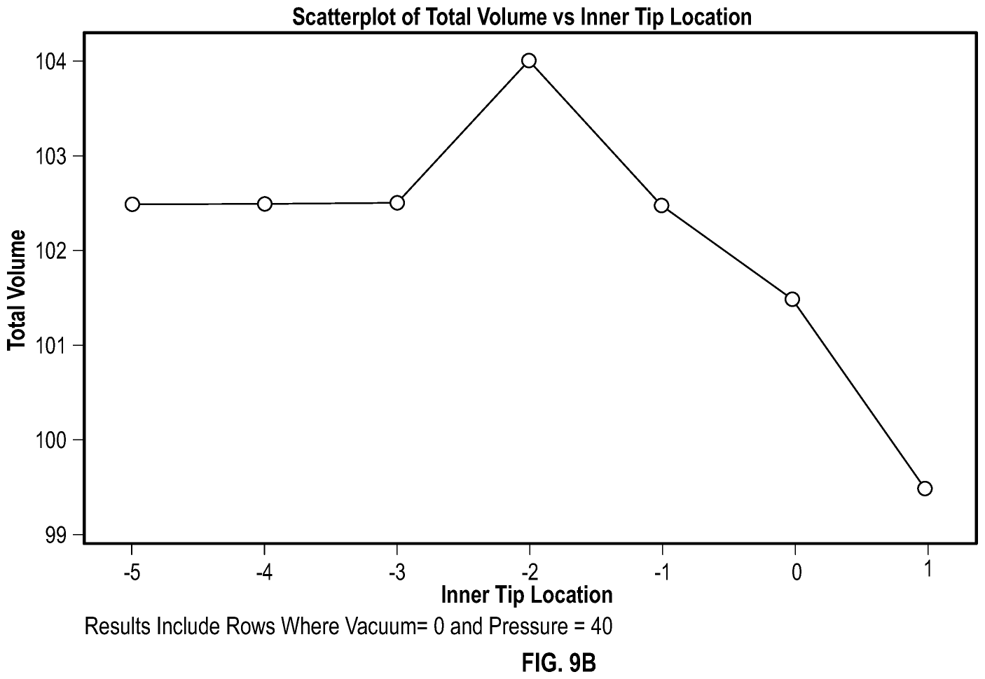

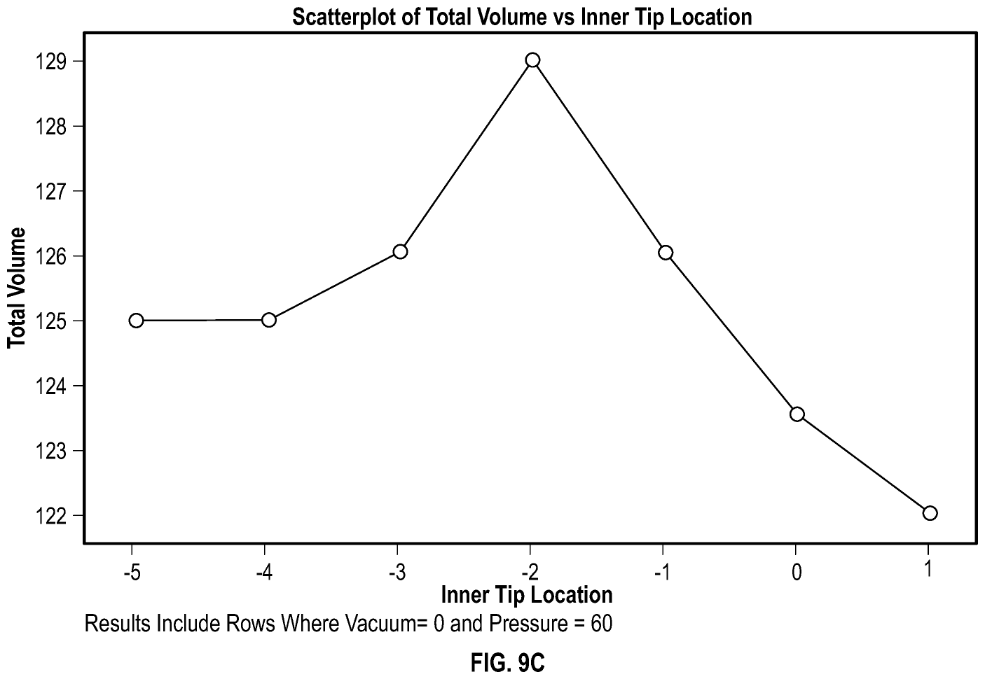

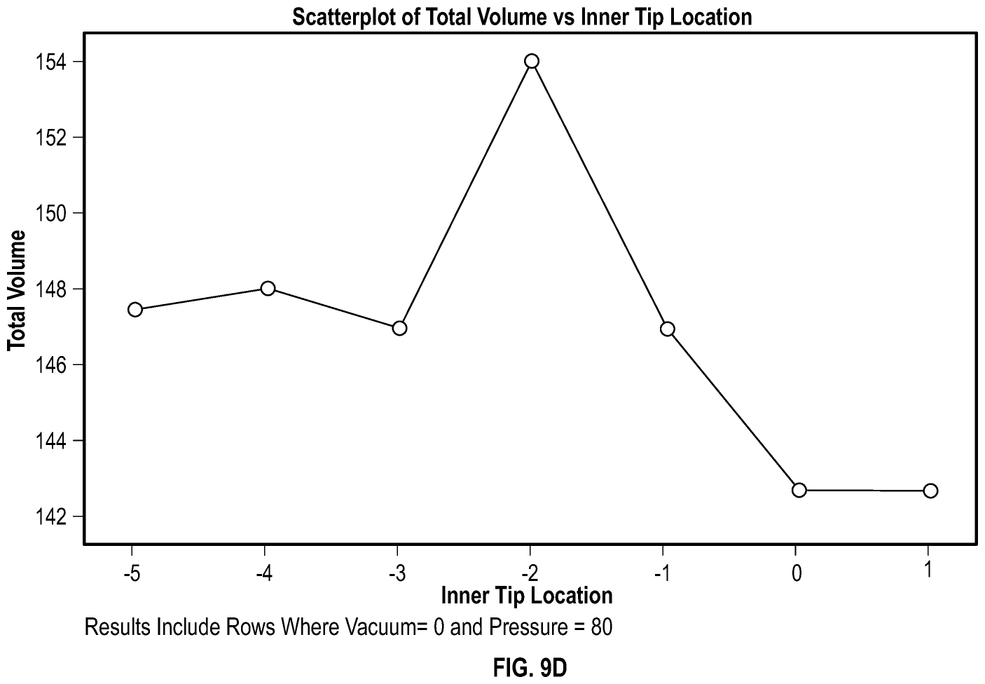

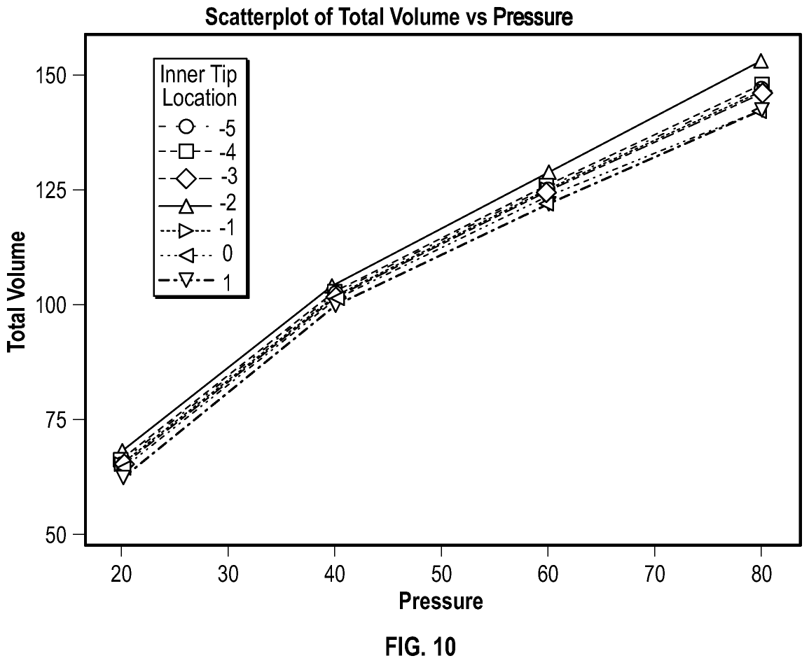

As shown in FIGS. 9A-9D, scatterplots of the total volume versus the inner tip location for each of the pressure conditions were compiled, as follows: 20 psi (FIG. 9A); 40 psi (FIG. 9B); 60 psi (FIG. 9C); and 80 psi (FIG. 9D). FIG. 10 is a scatterplot of the total volume versus the pressure.

Conclusion: As shown in FIGS. 9A-9D, the flow increases as the tip moves from a tip location of 1 to −2 before decreasing and appearing to reach a steady state. Total flow appears to be maximized at a tip location of −2.

### Example 3

Effect of Recirculation Ratio on Total Flow Rate in Different Return Lumen Inner Diameters at Various Infusion Flow Rates

Experiments were conducted to study the effect of recirculation ratio on total flow rate in different return lumen inner diameters (IDs) at various infusion flow rates using embodiments of the catheter system of the present disclosure.

As used herein, “Recirculation-to-Input Area Ratio” is defined as the cross-sectional area of the return lumen compared to the cross-sectional area of the input lumen.

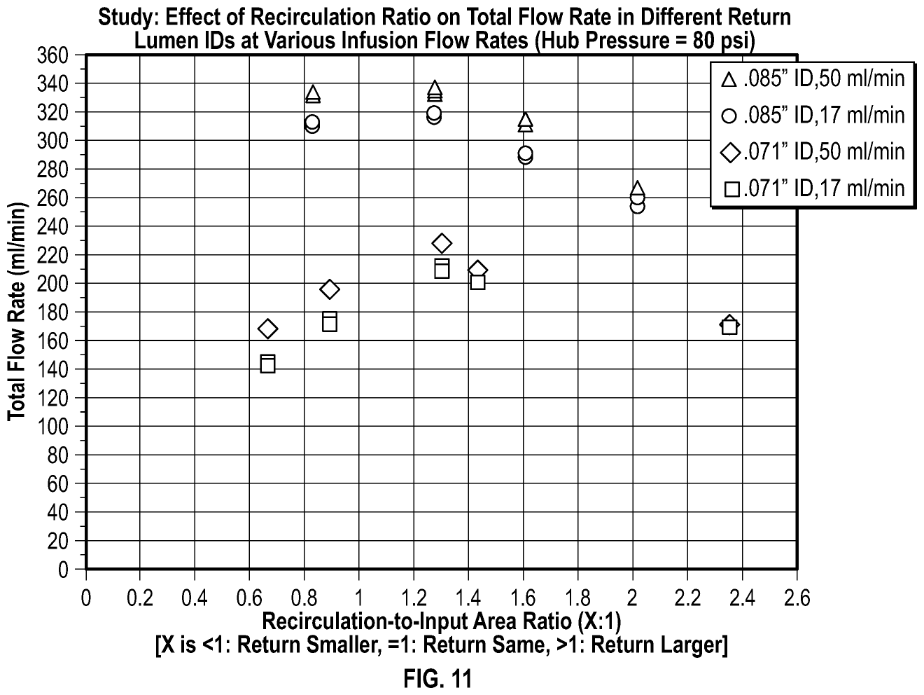

Results from the study are shown in FIG. 11, which is a graph illustrating the effect of recirculation ratio on total flow rate in different return lumen inner diameters at various infusion flow rates.

For catheters with an inner diameter of 0.085″ and smaller, a recirculation-to-input area ratio from 0.9 to 2.3 provides optimum total flow with the peak ratio occurring at 1.3.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Citation of a reference herein shall not be construed as an admission that such reference is prior art to the present invention. All references cited herein are hereby incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

Illustrative embodiments of the processes, methods, and products of the present disclosure are described herein. It should be understood, however, that the description herein of the specific embodiments is not intended to limit the present disclosure to the particular forms disclosed but, on the contrary, the intention is to cover all modifications, equivalents, and alternatives falling within the spirit and scope of the invention by the appended claims. Thus, although the present invention has been described for the purpose of illustration, it is understood that such detail is solely for that purpose and variations can be made by those skilled in the art without departing from the spirit and scope of the invention which is defined by the following claims.

1. A catheter system for infusing a chilled fluid into a subject, comprising:
an outer elongate member sized for insertion into a body cavity of a subject and having a transition section extending between, and in fluid communication with, a proximal section and a distal section of the outer elongate member, said distal section being an infusion lumen;
an inner elongate member extending within the proximal section of the outer elongate member and having an input lumen permitting fluid to flow in a forward direction from a proximal end of the inner elongate member to a distal end of the outer elongate member and into the body cavity; and
a return lumen extending within the proximal section of the outer elongate member and outside the input lumen and permitting fluid to flow in a reverse direction from the transition section to a proximal end of the outer elongate member,
wherein a lumen of the transition section provides sufficient flow resistance to urge a first portion of fluid flowing out of the input lumen in the forward direction to flow into the return lumen in the reverse direction while a second portion of the fluid flowing out of the input lumen in the forward direction flows out of the distal end of the outer elongate member.

2. The catheter system according to claim 1, further comprising a cooling device that cools (i) fluid entering the input lumen and (ii) fluid exiting the return lumen.

3. The catheter system according to claim 1, wherein a distal end of the inner elongate member is located in the proximal section of the outer elongate member.

4. The catheter system according to claim 1, wherein an inner diameter of the transition section lumen is smaller than an inner diameter of the input lumen.

5. The catheter system according to claim 1, wherein an inner diameter of the transition section lumen distally is smaller than an inner diameter of the transition section lumen proximally.

6. The catheter system according to claim 1, wherein an inner diameter of the transition section lumen tapers such that the inner diameter of the transition section lumen becomes progressively smaller distally than proximally.

7. The catheter system according to claim 1, wherein the body cavity is a blood vessel.

8. The catheter system according to claim 1, wherein the inner elongate member is unsecured within the outer elongate member.

9. The catheter system according to claim 1, wherein the return lumen is coaxial with the input lumen.

10. The catheter system according to claim 1, wherein the return lumen is parallel to the input lumen.

11. The catheter system according to claim 1, wherein a cross-sectional area of the return lumen is greater than a cross-sectional area of the input lumen.

12. The catheter system according to claim 1 further comprising a resistance member disposed within the input lumen and being effective to increase resistance to fluid flow within the input lumen.

13. The catheter system according to claim 12, wherein the resistance member is adjustable to vary resistance to fluid flow within the input lumen.

14. The catheter system according to claim 12, wherein the resistance member has multiple outer diameters and is adjustable such that positioning any of its outer diameters within the input lumen results in differing resistance to fluid flow within the input lumen.

15. The catheter system according to claim 12, wherein the resistance member comprises a wire that extends at least partially along the length of the input lumen.

16. The catheter system according to claim 1, wherein the respective cross-sectional areas of the return lumen and input lumen are configured using a recirculation-to-input area ratio parameter so as to provide a desired total flow rate of the catheter system, wherein the recirculation-to-input area ratio is defined as the cross-sectional area of the return lumen compared to the cross-sectional area of the input lumen.

17. The catheter system according to claim 16, wherein the recirculation-to-input area ratio ranges from about 0.9 to about 2.3 for catheters having an inner diameter of about 0.085 inches or less.

18. The catheter system according to claim 17, wherein the recirculation-to-input area ratio is about 1.3.

19. The catheter system according to claim 1 further comprising a placement mechanism to assist in positioning the inner elongate member within the outer elongate member at a desired position so as to affect a desired flow rate of the chilled fluid into the body cavity or a target region of the subject.

20. The catheter system according to claim 19, wherein the placement mechanism comprises marker bands fitted onto each of the inner elongate member and the outer elongate member to assist in positioning the inner elongate member within the outer elongate member at the desired position.

21. The catheter system according to claim 19, wherein the placement mechanism comprises a tubing stop fitted onto the inner elongate member to assist in positioning the inner elongate member within the outer elongate member at the desired position.

22. A method of infusing a chilled fluid into a subject in need thereof, said method comprising:
providing a catheter system according to claim 1;
inserting the distal end of the outer elongate member of the catheter system into the subject; and
infusing a chilled fluid into the subject through the input lumen of the catheter system.

23. A catheter system for use in infusing a chilled fluid into a subject in need thereof, wherein the catheter system according to claim 1 is provided, the distal end of the outer elongate member of the catheter system is inserted into the subject; and a chilled fluid is infused into the subject through the input lumen of the catheter system.

24. A cooling system for delivering a chilled fluid to an organ or tissue of a subject, comprising:
a catheter system according to claim 1; and
a chiller that cools fluid infused into the input lumen.

25. The cooling system according to claim 24 further comprising a saline source, a fluid delivery pump device, and an accumulator, wherein the chiller is a heat exchanger.

26. The cooling system according to claim 24 further comprising an elevated fluid source, a bubble trap, a waste container, and a fluid delivery pump device, wherein the chiller is a cooling unit that includes a bladder.

27. A method of delivering chilled fluids to an organ or tissue of a subject in need thereof, comprising:
providing a cooling system according to claim 24;
inserting the distal end of the outer elongate member of the cooling system into the body cavity of the subject; and
delivering a chilled fluid to an organ or tissue of the subject through the input lumen of the catheter system.

28. A cooling system for use in delivering chilled fluids to an organ or tissue of a subject in need thereof, wherein the cooling system according to claim 24 is provided, the distal end of the outer elongate member is inserted into a body cavity of the subject; and a chilled fluid is delivered to an organ or tissue of the subject through the input lumen of the catheter system.
